(19) United States
(12) Patent Application Publication
(10) Pub. No.: US 2010/0016901 A1
(43) Pub. Date: Jan. 21, 2010

(54) BONE SCREW RETAINING SYSTEM

(76) Inventor: James C. Robinson, Atlanta, GA (US)

Correspondence Address: Ballard Spahr LLP, SUITE 1000, 999 PEACHTREE STREET, ATLANTA, GA, 30309-3915, US

(21) Appl. No.: 11/922,299

(22) PCT Filed: Jun. 16, 2006
(86) PCT No.: PCT/US2006/023377

§ 371 Date: Oct. 1, 2009

Related U.S. Application Data
(60) Provisional application No. 60/691,318, filed on Jun. 16, 2005.
(60) Provisional application No. 60/785,453, filed on Mar. 24, 2006.

(51) Int. Cl. A61B 17/80

(52) U.S. Cl. 606/289; 606/286

(57) ABSTRACT

A bone screw retaining system including an implant adapted to be applied the anterior human cervical spine for contacting anterior aspects of at least two cervical vertebral bodies, the implant having a plate that defines a plurality of transversely extending bores that extend along a longitudinal axis between an anterior surface and a posterior surface, each bore being configured to receive a bone screw for engaging the plate to the cervical spine. An elastically deformable spring member positioned therein a cavity of the plate such that portions of the spring member can extend into a portion of an upper region of each of the bores of a pair of opposing bores, the spring member being movable between a first relaxed, expanded position and a second, compressed position. In the first relaxed position, at least a portion of the spring member extends outwardly substantially transverse to the longitudinal axis of the bore and into the upper region of the bore and, in the second position, portions of the spring member are medially biased away from the longitudinal axis of the bore toward the outer wall of upper region of the bore, which increases the effective inner diameter of the upper region of the bore.

### FIELD OF THE INVENTION

The present invention relates generally to surgical procedures, most particularly for use in fixation of the cervical spine. More particularly, the invention pertains to a bone screw retaining system for use in a plate system for anteriorly fixating adjacent cervical vertebrae.

### BACKGROUND OF THE INVENTION

As with any bony structure, the spine is subject to various pathologies that compromise its load bearing and support capabilities. The spine is subject to degenerative diseases, the effects of tumors and, of course, fractures and dislocations attributable to physical trauma. In the past, spinal surgeons have tackled the thorny problems associated with addressing and correcting these pathologies using a wide variety of instrumentation and a broad range of surgical techniques. For example, in spinal surgeries, the fusion of two or more vertebral bodies is required to secure a portion of the spinal column in a desired position. Alternatively, the use of elongated rigid plates has been helpful in the stabilization and fixation of the lower spine, most particularly the thoracic and lumbar spine.

The cervical spine can be approached either anteriorly or posteriorly, depending upon the spinal disorder or pathology to be treated. Many of the well known surgical exposure and fusion techniques of the cervical spine are described in Spinal Instrumentation, edited by Drs. Howard An and Jerome Cotler. This text also describes instrumentation that has been developed in recent years for application to the cervical spine, most frequently from an anterior approach.

The anterior approach to achieving fusion of the cervical spine has become the most popular approach. During the early years of cervical spine fusion, the fusions were preformed without internal instrumentation, relying instead upon external corrective measures such as prolonged recumbent traction, the use of halo devices or minerva casts, or other external stabilization. However, with the advent of the elongated plate customized for use in the cervical spine, plating systems have become the desired internal stabilization device when performing stabilization operations.

It has been found that many plate designs allow for a uni-corticaly or bi-corticaly intrinsically stable implant. It has also been found that fixation plates can be useful in stabilizing the upper or lower cervical spine in traumatic, degenerative, tumorous or infectious processes. Moreover, these plates provide the additional benefit of allowing simultaneous neural decompression with immediate stability.

During the many years of development of cervical plating systems, particularly for the anterior approach, various needs for such a system have been recognized. For instance, the plate must provide strong mechanical fixation that can control movement of each vertebral motion segment in six degrees of freedom. The plate must also be able to withstand axial loading in continuity with each of the three columns of the spine. The plating system must be able to maintain stress levels below the endurance limits of the material, while at the same time exceeding the strength of the anatomic structures or vertebrae to which the plating system is engaged.

Further plating systems also typically require the thickness of the plate to be small to lower its prominence, particularly in the smaller spaces of the cervical spine. Additionally, the screws used to connect the plate to the vertebrae must not loosen over time or back out from the plate. This requirement, that the bone screws do not loosen over time or back out from the plated, tends to complicate implantation of known plating systems. Such bone screw retention systems generally ensure that the bone screws placed into the vertebrae through the plating system do not back out voluntarily from the plate, but typically do not adequately permit the removal of an associated bone screw when desired by the surgeon.

On the other hand, while the plate must satisfy certain mechanical requirements, it must also satisfy certain anatomic and surgical considerations. For example, the cervical plating system must minimize the intrusion into the patient and reduce the trauma to the surrounding soft tissue. It is known that complications associated with any spinal procedure, and most particularly within the tight confines of cervical procedures, can be very devastating, such as injury to the brain stem, spinal cord or vertebral arteries. It has also been found that optimum plating systems permit the placement of more than one screw in each of the instrumented vertebrae.

More specifically, it is known that bone screws can be supported in a spinal plate in either a rigid or a semi-rigid fashion. In a rigid fashion, the bone screws are not permitted any micro-motion or angular movement relative to the plate. In the case of a semi-rigid fixation, the bone screw can move somewhat relative to the plate during the healing process of the spine. It has been suggested that semi-rigid fixation is preferable for the treatment of degenerative diseases of the spine. In cases where a graft is implanted to replace the diseased vertebral body or disk, the presence of a screw capable of some rotatation ensures continual loading of the graft. This continual loading avoids stress shielding of the graft, which in turn increases the rate of fusion and incorporation of the graft into the spine.

Similarly, rigid screw fixation is believed to be preferable in the treatment of tumors or trauma to the spine, particularly in the cervical region. It is believed that tumor and trauma conditions are better treated in this way because the rigid placement of the bone screws preserves the neuro-vascular space and provides for immediate stabilization. It can certainly be appreciated in the case of a burst fracture or large tumorous destruction of a vertebra that immediate stabilization and preservation of the vertebral alignment and spacing is essential. On the other hand, the semi-rigid fixation is preferable for degenerative diseases because this type of fixation allows for a dynamic construct. In degenerative conditions, a bone graft is universally utilized to maintain either the disc space and/or the vertebral body itself. In most cases, the graft will settle or be at least partially resorbed into the adjacent bone. A dynamic construct, such as that provided by semi-rigid bone screw fixation, will compensate for this phenomenon.

Furthermore, known plating systems often do not permit sufficient angular freedom for bone screws relative to the plate. Generally, known plating systems have defined bores through which bone screws are placed at a predefined angle. Therefore, the operating surgeon often does not have freedom to insert the bone screws into the vertebrae as to best fit the anatomy of the individual patient. While some known systems do permit bone screw angulation, they typically are not adapted to be used with an easy to use bone screw retaining mechanism.

It remains desirable in the pertinent art to provide a bone screw retaining system for use with a plating system that addresses the limitations associated with known systems, including but not limited to those limitations discussed above.

### SUMMARY

In one embodiment of the present invention, a bone screw retention system comprises a plate having a plurality of bores therein and a plurality of spring members mounted therein the upper portion of each bore

Related methods of operation are also provided. Other systems, methods, features, and advantages of the bone screw retention system will be or become apparent to one with skill in the art upon examination of the following figures and detailed description. It is intended that all such additional systems, methods, features, and advantages be included within this description, be within the scope of the bone screw retention system, and be protected by the accompanying claims.

### DESCRIPTION OF THE FIGURES

The accompanying drawings, which are incorporated in and constitute a part of this specification, illustrate certain aspects of the instant invention and together with the description, serve to explain, without limitation, the principles of the invention.

FIG. 1 is a top plan view of a first embodiment of the present invention showing a bone screw retention system comprising a plate having a plurality of bores therein and a plurality of spring members shown in a locking position with a plurality of seated bone screws.

FIG. 2A is a perspective view of the bone screw retention system of FIG. 1, showing an exemplary bone screw being inserted therein a bore of the plate.

FIG. 2B is a perspective view of the bone screw retention system of FIG. 1, showing the bone screw seated therein the bore of the plate.

FIG. 3 is a perspective view of the bone screw retention system of FIG. 1, showing a plurality of bone screws seated therein the bores of the plate and positioned at desired angles relative to the plate.

FIG. 4 is a perspective view of the plate of the bone screw retention system of FIG. 1, showing a spring member comprising a split-ring operable mounted in a spring mount such that, in a first relaxed position, a portion of the split-ring extends over a portion of the upper region of the bore.

FIG. 5 is a perspective view of the spring member of the bone screw retention system of FIG. 4.

FIG. 6 is a perspective view of the spring mount of the bone screw retention system of FIG. 4.

FIG. 7A is a perspective view of a bone screw being initially placed therein the bore of the plate.

FIG. 7B is a perspective view of the bone screw being advanced into the underlying bone, showing the spring member being deflected medially by the taper of the head of the bone screw which allows it to pass by the spring member.

FIG. 7C is a perspective view of the bone screw as it is advances sufficiently past the spring member such that the spring member biases back to its original relaxed position in which at least a portion of the spring member overlies a portion of the now underlying bone screw.

FIG. 8 is a partial cross-sectional view of a second embodiment of the present invention showing a plurality of spring members mounted therein the upper portion of each bore of the plate; each spring member comprising a spring assembly that comprises a movable piston member biased by a coil spring.

FIG. 9 is a partial top plan view of the bone screw retention system of FIG. 8.

FIG. 10 is a partial cross-sectional view of a third embodiment of the present invention showing at least one spring member mounted therein the upper portion of each bore of the plate; each spring member comprising an arcuate spring member mounted therein the wall of the upper region of the bore and which is shown in its first, relaxed position.

FIG. 11 is a partial top plan view of the bone screw retention system of FIG. 10.

FIG. 12 shows a tubular screw drive guide being inserted onto a screw driver.

FIG. 13 is an enlarged partial perspective view of the distal end of the screw drive guide, showing a shoulder surface formed at the distal end of the screw drive guide that is configured to engage the spring member.

FIG. 14 is a perspective view showing the screw drive guide being placed on a top portion of the seated screw and rotated such that the shoulder surface contacts and deflects medially the spring member.

FIG. 15 is a perspective view showing the screwdriver being rotated to back out the screw while the distal end of the screw drive guide remains in contact with a top portion of screw.

FIG. 16 is a partial perspective view of a distal end of a tubular screw driver guide having at least one recess adapted to fit over the spring member in its first, relaxed position.

FIG. 17 is a partial perspective view of a screw removal assembly shown in a first position in which a distal end portion of a screw drive member extends outwardly and away from the distal end of a sleeve member, the distal end portion configured to operatively engage a head of a bone screw.

FIG. 18 is a partial fragmentary perspective view of the screw removal assembly of FIG. 17 shown in a second position in which the sleeve member is pushed downwardly relative to the screw drive member, against the resistance of a bias member, such that the distal end portion of the screw drive member is enclosed by the distal end of the sleeve member, the distal end of the sleeve member having at least one recess adapted to fit over the spring member in its first, relaxed position.

FIG. 19 is a partial perspective view of a screw removal assembly shown in a first position in which a distal end portion of a screw drive member is positioned within, and at least partially enclosed by, the distal end of a sleeve member, the distal end of the sleeve member having at least one recess adapted to fit over the spring member in its first, relaxed, position.

FIG. 20 is a partial fragmentary perspective view of the screw removal assembly of FIG. 19 shown in a second position in which the sleeve member is pushed upwardly relative to the screw drive member, against the resistance of a bias member, such that the distal end portion of the screw drive member extends outwardly and away from the distal end of a sleeve member, the distal end portion configured to operatively engage a head of a bone screw.

### DESCRIPTION OF THE INVENTION

The present invention can be understood more readily by reference to the following detailed description, examples, and claims, and their previous and following description. Before the present system, devices, and/or methods are disclosed and described, it is to be understood that this invention is not limited to the specific systems, devices, and/or methods disclosed unless otherwise specified, as such can, of course, vary. It is also to be understood that the terminology used herein is for the purpose of describing particular aspects only and is not intended to be limiting.

The following description of the invention is provided as an enabling teaching of the invention in its best, currently known embodiment. Those skilled in the relevant art will recognize that many changes can be made to the embodiments described, while still obtaining the beneficial results of the present invention. It will also be apparent that some of the desired benefits of the present invention can be obtained by selecting some of the features of the present invention without utilizing other features. Accordingly, those who work in the art will recognize that many modifications and adaptations to the present invention are possible and can even be desirable in certain circumstances and are a part of the present invention. Thus, the following description is provided as illustrative of the principles of the present invention and not in limitation thereof.

As used herein, the singular forms “a,” “an” and “the” include plural referents unless the context clearly dictates otherwise. Thus, for example, reference to a “bore” includes aspects having two or more bores unless the context clearly indicates otherwise.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

As used herein, the terms “optional” or “optionally” mean that the subsequently described event or circumstance may or may not occur, and that the description includes instances where said event or circumstance occurs and instances where it does not.

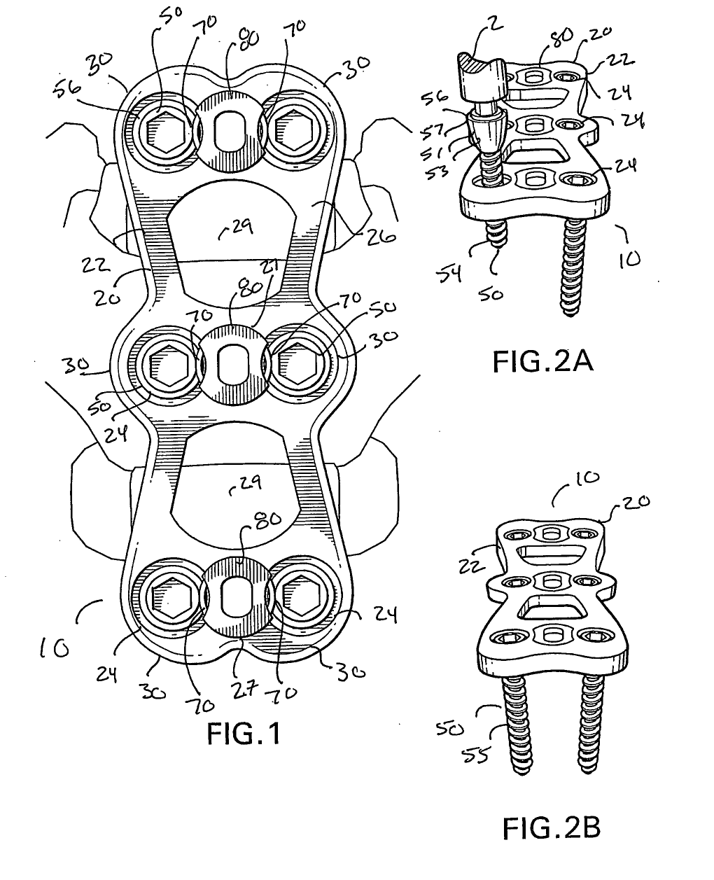

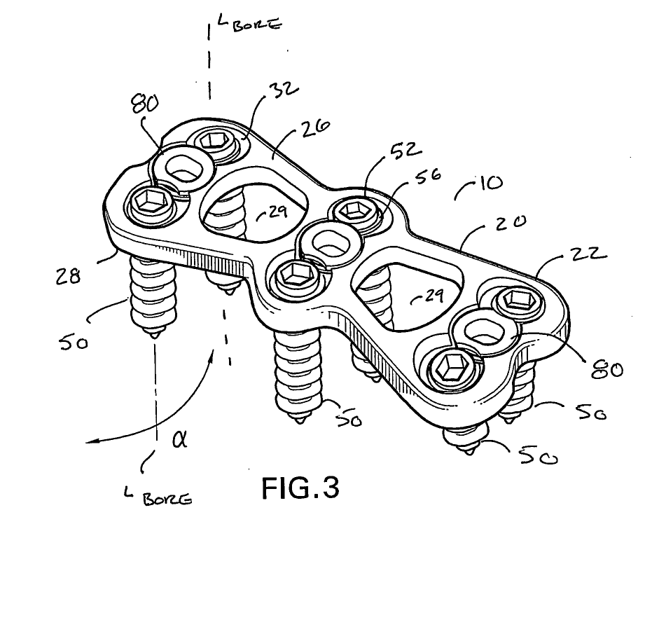

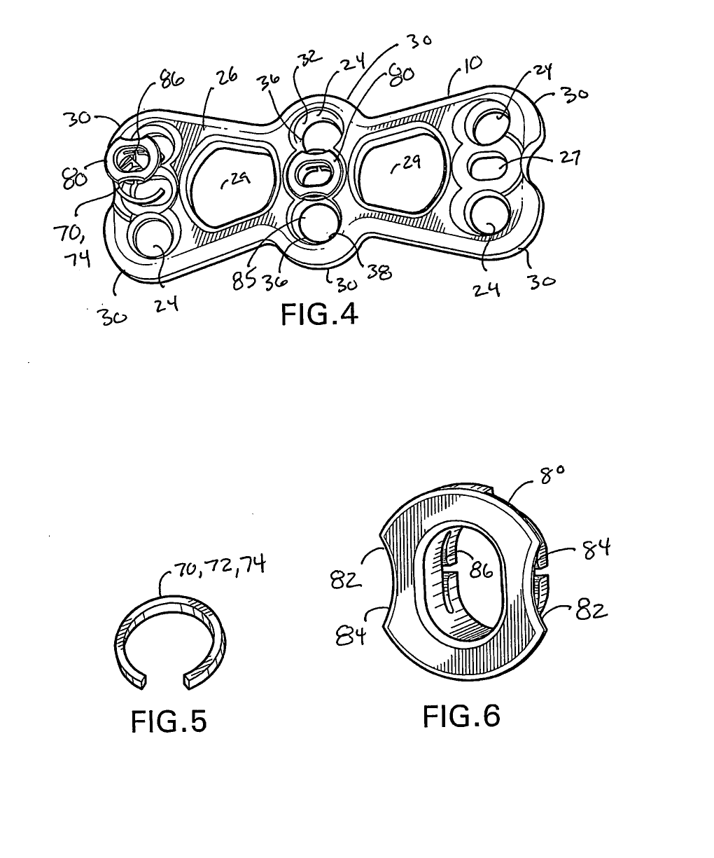

In one embodiment, and referring to FIGS. 1-7A, the present invention comprises an implant 10, particularly for the spinal column, comprising a joining member 20 such as a plate 22 that defines a plurality of openings or bores 24, bone screws 50 capable of being accommodated in the bores, and at least one spring member 70 configured for releasably securing the bone screws therein the bores. In one aspect, the spring member can come into direct contact with the bone screw to secure the bone screw within the bores. Optionally, the spring member can form a blocking element to secure the bone screw within the bores. Further, the system of the present invention provides for the selective removal of the bone screw or screws from the plate at the physicians desire.

In one aspect, the joining member 20 comprises a plate 22 that defines a plurality of transversely extending bores 24 that are counter sunk a predetermined distance. In one exemplary aspect, a head 52 of a bone screw 50 can be configured to be posteriorly displaceable through a bore 24 of the plate from an anterior surface 26 to a posterior surface 28 of the plate and retained within a portion of the bore between the posterior and anterior surfaces 26, 28. In one aspect, the plate 22 can have a generally elongated form whose outline generally departs from rectangular due to the presences of partial lobes 30 or lateral projections at the corners and at the center of the sides of the plate. Each partial lobe 30 has a rounded outline and, in an exemplary aspect, can define one respective bore 24. It is, of course, contemplated that other shapes of the plate may be employed.

As noted above, the plate defines a plurality of bores 24 that extend substantially transverse therethrough the plate between the anterior and posterior surfaces 26, 28 of the plate and that are configured for operable receipt of the bone screw or bone anchor. In one aspect, the bores 24 extend along a longitudinal axis from the anterior surface to the bottom bone contacting posterior surface of the plate. In one aspect, each bore 24 has an upper region 32 with a first diameter and a lower region 34 that includes a seat 36 for the bone screw and a posteriorly extending tubular shaft 38 that extends to an opening on the posterior surface 28 of the plate 22. In one aspect, the seat 36 of the bore can have at least a partial spherical shape. In another aspect, the bores comprise a plurality of paired opposing bores

In a further aspect, the bone screw 50 has a head 52 with a maximum diameter that is smaller than the first diameter of the upper region 32 of the bore, which thereby allows the screw head to pass through that region of the bore. In one example, the bone screw can be a conventional self tapping bone screw. It is of course contemplated that conventional non self tapping bones screws can be used with the system of the present invention. Further, it is contemplated that conventional bone screws with at least partially rotatable heads can be used if a semi-rigid fixation procedure is desired.

In various exemplary aspects, the head 52 of each bone screw 50 can comprise a complementary tapered section 51 that extends outwardly therefrom the threaded shank portion 54 of the bone screw. In this aspect, the tapered section 51 can have a tapered surface 53 that extends from a shank 55 of the bone screw toward an upwardly facing shoulder surface 56 of the bone screw which is formed by a portion of the shoulder 57 of the head of the bone screw. A portion of the bone screw above the upwardly facing shoulder surface 56 of the bone screw is conventionally configured for operative engagement with a driving tool 2 and has a reduced diameter relative to the diameter of the shoulder of the head of the bone screw.

In one aspect, the seat 36 is configured for complementary receipt of the shank 55 of the bone screw 50 such that the bone screw can be fixed at a predetermined angle with respect to the plate. Alternatively, the bone screw can be fixed at an operator selective angle, i.e., be angularly displaceable. In one example, the tapered section 51 of the bone screw can be configured for complementary rotatable contact with an exemplary spherically shaped seat of the bore. It is contemplated that the tapered section 51 of the bone screw can be substantially linear or, optionally, substantially spherical. Further, the shank 55 of the bone screw can be threaded in any well known fashion and may include an axial groove to enable the bone screw to be self-boring and self-tapping.

In another aspect, the shaft 38 of the bore can have an operative diameter that is greater than the diameter of the shank 55 of the bone screw intermediate the head of the bone screw and its distal end. As a result, the bone screw 50 is angularly displaceable within the shaft of the bore between the seat and the posterior surface opening. The bone screw can thus be tilted within the shaft 38 of the bore relative to the longitudinal axis of the bore to facilitate positioning the bone screw 50 at a desired location in the bone by advancing the threaded shank portion 54 of the bone screw within the bone at an angle relative to the posterior surface of the plate. In one aspect, the bone screw 50 can be angularly displaced relative to the longitudinal axis of the bore up to an angle α of about 20 degrees. Thus, the surgeon has, at his disposal, the freedom to orient the bone screw angularly with respect to the joining member or plate, which allows him to optimize the anchorage. In one aspect, the bone screws can be rotatably mounted therein the underlying bone tissue using a conventional screw driver, a drive socket, and the like.

In one embodiment of the present invention, the spring member 70 comprises a circlip 72. In one exemplary aspect, the circlip 72 is in the form of a circular split-ring 74 having spaced opposed ends. In one embodiment, at least portions of one circlip is common to two bores 24 in the plate 22, for example, the two bores 24 forming a pair of opposing bores. In an alternative embodiment, at least a portion of one respective circlip is common to one individual bore 24 of the plate 22. As used herein, the terms “circlip” and “split-ring” are used interchangeably without intended limitation. As described herein, it is contemplated that the exemplified plate, bone screws and split-rings may be supplied as part of a bone screw retaining system for use by a surgeon.

In one embodiment, the plate 22 further defines a plurality of cavities 27. In one exemplary aspect, at least a portion of the cavity 27 forms a transversely extending cavity that opens on both the posterior and anterior surfaces of the plate. In another aspect, the plurality of cavities can be spaced substantially along the longitudinal axis of the plate 22. In a further aspect, one cavity 27 is positioned therebetween each pair of bores 24. In this aspect, it is contemplated that the cavity can be positioned adjacent to and equidistant from each bore of the respective pair of bores.

In another aspect, one spring member 70, e.g., one split-ring 72, is operable positioned therein each cavity 27 such that portions of the spring member can extend into a portion of the upper region of each of the bores of the paired opposing bores. In this aspect, the elastically deformable spring member 70 is configured to mount therein the cavity 27 and is movable between a first relaxed, expanded position and a second, compressed position. In one aspect, the spring member 70 is mounted to extend outwardly substantially transverse to the longitudinal axis of the bore and into the upper region of the bore. As one will appreciate, in the second position, the spring member 70 has a diameter that is less than the diameter of the spring member when it is in the first, relaxed position. Further, in this aspect, when the spring member 70 is in the first relaxed position, portions of the spring member extend over portions of the upper region 32 of each bore of the paired opposing bores, which deceases the effective inner diameter of the upper region 32 of the bore. In another aspect, when the spring member 70 is in the second, compressed position, portions of the spring member 70 are medially biased away from the longitudinal axis of the bore 24 toward the diameter of the outer wall of upper region 32 of the bore. As one will appreciate, the effective inner diameter of the upper region 32 of the bore is thereby increased when the spring member 70 is in the second position.

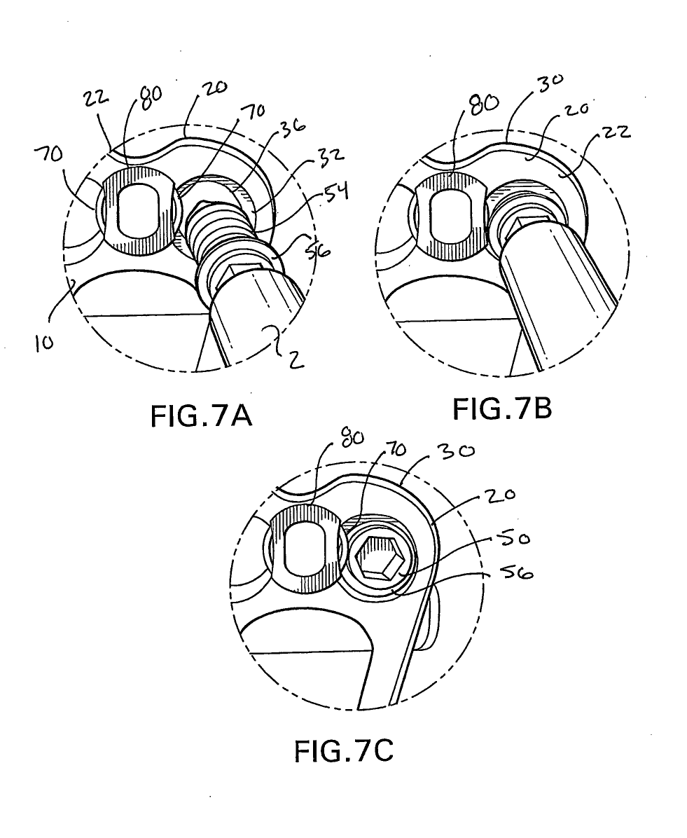

In one exemplified embodiment of the present invention, and as shown in FIGS. 7A-7C, the split-ring 74 is mounted therein the cavity 27 and the bone screw is inserted therein the bore of the plate (shank first into the plate from the anterior surface of the plate) and is advanced posteriorly within the bore 24. The tapered section 51 of the head 52 of the bone screw engages the split-ring and applies a radially expanding force against a peripheral surface of the split-ring to forcefully move the split-ring medially from the first position toward the second, compressed position. One would appreciate that the interaction between the split-ring 74 and the head 52 of the bone screw causes the effective inner diameter of the upper region 32 of the bore to increase to a size that allows for the posterior passage of the head of the bone screw past the split-ring. In this aspect, after the shoulder 57 of the head of the bone screw passes the operative plane of the spring member, the split-ring biases medially back to its first relaxed position such that a portion of the split-ring overlies a portion of the upwardly facing shoulder surface 56 of the head of the bone screw. Thus, when the split-ring 74 relaxes to its unexpanded state, it prevents the bone screw 50 from backing out of the plate as the effective inner diameter of the upper region 32 of the bore is less than the diameter of the head of the bone screw, which effectively blocks the path that the bone screw would have to traverse to back out or exit the bore in the plate. With the bone screw 50 positioned against the seat 36 of the bore, the distal threaded portion of the bone screw is embedded in, and secured to, the bone of the patient.

In one aspect, when the head 52 of the bone screw fully engages the seat 36 of the plate, the upwardly facing shoulder surface 56 of the bone screw is located at or below the substantially transverse plane of the spring member 70. In this position, as one will appreciate, the spring member biases back toward and/or to its relaxed position because the portion of the bone screw above the plane of the spring member has a reduced diameter relative to the upwardly facing surface portion of the bone screw.

In one aspect, the system of the present invention further comprises a spring mount 80 adapted to fixedly mount therein the cavity. In a further aspect, the spring mount is configured for a compressive fit within the cavity 27. In another aspect, a portion of opposing side walls 82 of the spring mount is recessed such that the upper region of the bore and the recessed edge portion 84 of the spring mount define a generally circular countersunk well 85 that is sized to receive the bone screw. In the relaxed position, a portion of the spring member 70, 74 spans across a portion of the recessed edge portion 84 and extends outwardly over a portion of the countersunk well substantially transverse to the longitudinal axis of the bore. In one aspect, the spring mount 80 has a groove 86 and/or slot defined therein the side walls of the spring mount, which is configured to receive a portion of the spring member 70 as it is medially biased toward its second, compressed position when the bone screw is being inserted therein the bore of the plate. In a further aspect, the opposed ends of the spring clip can be enclosed therein the spring member.

In one embodiment, the spring member is formed from a biocompatible, flexible material such as, and not meant to be limiting, titanium alloy and the like as disclosed in U.S. Pat. Nos. 4,857,269 and 4,952,236, which are incorporated in their entirety herein by reference. Further, polymeric materials such as, for example, ultra-high molecular weight polyethylene can also be used to form the spring member of the present invention.

In another aspect, the plate can define a pair of opposing openings 29. In this aspect, the pair of openings 29 is generally positioned on the longitudinal axis of the plate. In a further aspect, each opening 29 is positioned intermediate the center and an end of the plate. Thus, in an embodiment having bores 24 in each of the partial lobes at the corners of the end of the plate and in the partial lobes at the center of the plate; it is contemplated that the opening can be positioned substantially between the respective bores. Thus, in this aspect, the plate forms a substantially open frame. These opposed openings 29 allow for visualization of the underlying bone and tissue as the implant is being fixated.

In one aspect, the plate 22 may be curved to match the anatomical curvatures. Thus, the implant curved to best suit the anatomy and natural curvature of the spinal column in the case of a spinal application. Of course, the plate 22 may be used in fracture fixation, as a tibial base plate, as a hip side plate or any application where bone plates and screws are used. For these uses, a larger screw than that described herein is necessary. Thus, it is contemplated that the screw locking system of the present invention can be scaled up or down as necessary so that any size screw can be utilized.

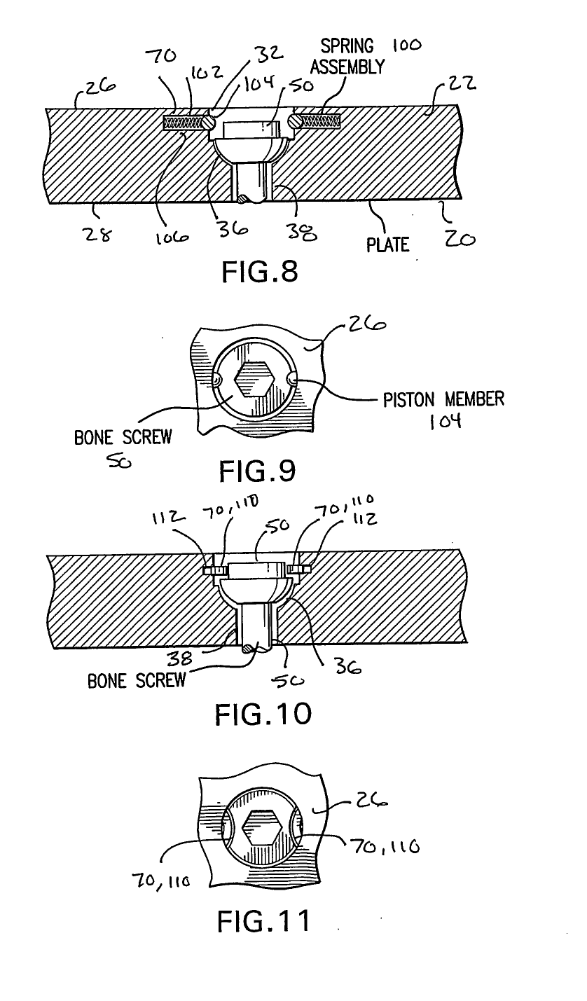

Referring to FIGS. 8 and 9, in another embodiment, the spring member 70 comprises at least one spring assembly 100 that comprises a coil spring 102 and a piston member 104. In this embodiment, the spring assembly is mounted therein a portion of the bore 24 such that in a relaxed position, a portion of the piston member 104 of the spring assembly extends over a portion of the upper region of the bore. In this aspect, a portion of the wall of the upper region of the bore defines an orifice 106 that is adapted to moveably receive the coil spring and at least a portion of the piston member therein. As one skilled in the art will appreciate, the piston member 104 is captured therein the orifice 106 such that it can not be ejected from the orifice by the urging of the coil spring. Thus, the coil spring of the spring assembly is positioned therein a portion of the wall of the upper region 32 of the bore. Of course, it is contemplated that a plurality of spring assemblies can be mounted in each bore of the plate.

In use, upon insertion of the bone screw into the bore of the plate and its subsequent posterior movement, the tapered surface 53 of the head 52 of the bone screw acts on the piston member 104 to force the piston member back into orifice by acting on, i.e., compressing, the underlying coil spring. In one aspect, when the head 52 of the bone screw fully engages the seat 36 of the plate, the upwardly facing shoulder surface of the bone screw is located at or below the plane of the piston member. In this position, as one will appreciate the coil spring 102 acts on the piston member 104 and bias the piston member outwardly toward and/or to its fully extended position.

In a further embodiment, and as shown in FIGS. 10 and 11, the spring member 70 can be at least one arcuate spring member 110 that is mounted to a portion of the upper region 32 of the bore such that in a relaxed position, a portion of the arcuate spring member 110 extends over a portion of the upper region of the bore substantially transverse to the longitudinal axis of the bore. A portion of the wall of the upper region of the bore can define a groove 112 and/or slot that is adapted to receive a portion of the arcuate spring member 70 as it is biased toward its second, compressed position when the bone screw is being inserted therein the bore of the plate. In a further aspect, the respective ends of the arcuate spring member 110 are mounted therein a portion of the wall of the upper region of the bore. Of course, it is contemplated that a plurality of arcuate spring members can be mounted in each bore of the plate.

Similar to the embodiment described above, in use, upon insertion of the bone screw into the bore 24 of the plate and it subsequent posterior advancement, the tapered surface of the head of the screw 50 acts on the arcuate spring member 110 to force the arcuate spring member toward its second, compressed position. In one aspect, when the head of the bone screw fully engages the seat 36 of the plate, the upwardly facing surface of the bone screw is located at or below the plane of the spring member 70. In this position, as one will appreciate, the arcuate spring member 110 biases toward and/or to its relaxed position because the portion of the bone screw above the plane of the spring member has a reduced diameter relative to the upwardly facing shoulder surface portion of the bone screw.

Also envisaged is a method for implanting the implant involving accessing the spinal column via an anterior route, fitting the implant, preparing the anchorage, fitting the anchorage members, locking the implant and the head of the anchoring members with respect to the joining member, and closing up the access route.

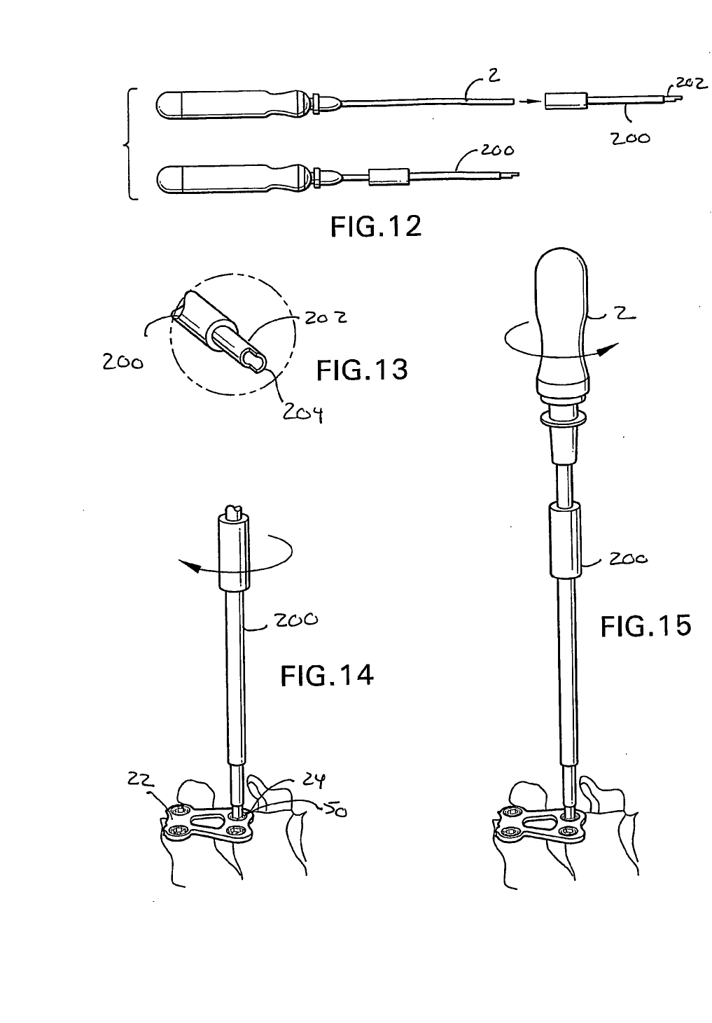

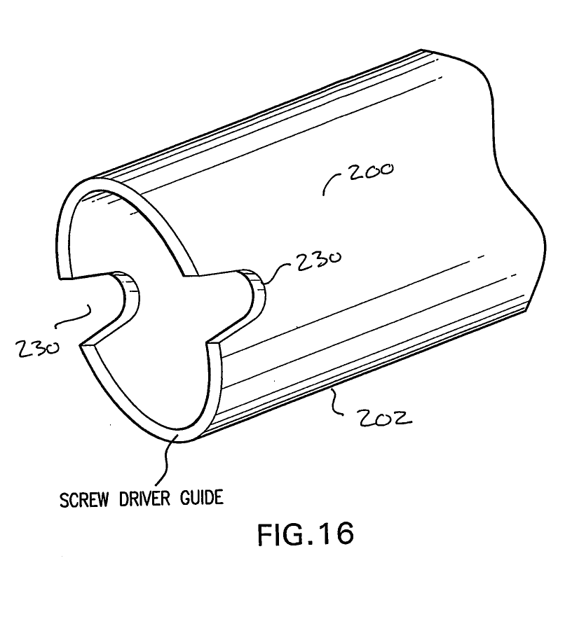

Referring generally to FIGS. 12-20, the bone screw retaining system of the present system contemplates the selective removal of the bone screw from the plate. In one embodiment, shown in FIGS. 12-15, the bone screw retaining system further comprises a tubular screw driver guide 200. In this embodiment, the screw driver guide has a distal end 202 that defines an arcuate shoulder surface 204 that is configured to engage the spring member. In use, rotation of the screw driver guide when seated therein the upwardly facing shoulder surface of the bone screw forces the shoulder surface 204 of the screw driver guide to come into contact with the spring member 70 and to force the spring member into or toward the second compressed position. The subsequent rotation of an appropriately sized screw driver is passed through the conduit of the screw driver guide backs moves both the screw driver and the bone screw in an anterior direction such that the spring member biases back to contact with the tapered portion of the screw as it is anteriorly moved.

In another embodiment shown in FIG. 16, the upper region of the formed bores 24 in the plate 22 have a diameter that is greater than the diameter of the shoulder 57 of the bone screw 50 such that a predetermined spaced is formed between the shoulder of the bone screw and the wall of the upper region 36 of the bore when the bone screw is positioned on the seat of the bore. In this embodiment, the tubular screw driver guide 200 is adapted to seat therein the predetermined space such that the removal of the bone screw is not obstructed.

In a further aspect, the screw driver guide 200 has a distal end 220 that defines at least one recess 230 that is adapted to extend over and about the spring member 70 of the bone screw retention system when it is in its first relaxed position. In use, the rotation of the screw driver guide 200 when seated therein the bore forces the outside wall of the screw driver guide to come into contact with the spring member and subsequently forces the spring member into or toward the second compressed position. Thus, when positioned, the screw driver guide defines a conduit that is sized to accommodate the bone screw. An appropriately sized screw driver is passed through the conduit and the bone screw is subsequently removed. When the screw driver guide 200 is removed from the bore of the plate, the spring member biases back to its first relaxed position.

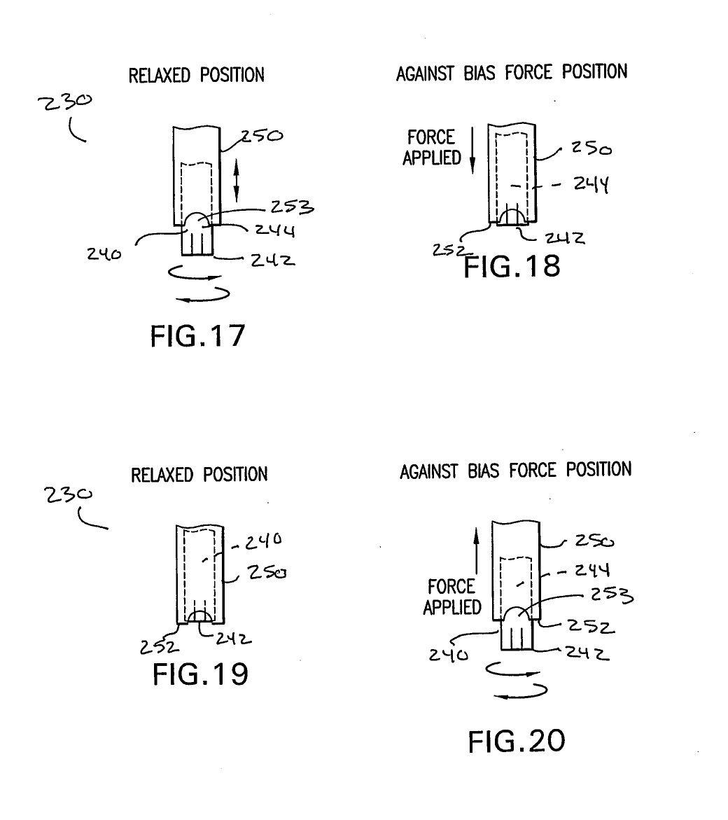

Referring now to FIGS. 17-20, the bone screw retaining system of the present invention can further comprise bone screw removal assemblies 230 that are configured to selectively remove bone screws 50 from the plate 22. In one aspect, the bone screw removal assembly 230 comprises an elongated screw drive member 240 and a sleeve member 250. In one aspect, the screw drive member has a distal end 242 that is configured to operatively engage the head 52 of a bone screw. In another aspect, the sleeve member 250 is configured to move longitudinally relative to and about the elongate screw drive member.

In one embodiment, and referring to FIGS. 17 and 18, in a first, relaxed position, the distal end portion 244 of the screw drive member extends outwardly and away from the distal end 252 of the sleeve member. The screw removal assembly can be moved to a second position, in which the sleeve member 250 is pushed downwardly longitudinally relative to the screw drive member 240 against the resistance of a bias member (not shown) operative positioned therebetween the screw drive member and the sleeve member. In the second position, the distal end portion 244 of the screw drive member is at least partially enclosed by the distal end 252 of the sleeve member. The distal end 252 of the sleeve member has at least one recess 253 that is adapted to fit over the spring member 70 in the spring member's first, relaxed position.

In use, the distal end portion 244 of the screw drive member 240 can be positioned into engagement with the head 52 of the bone screw and the sleeve member is subsequently pushed down and into a seated position in the bore 24. The rotation of the sleeve member 250 when it is in the seated position forces the outside wall of the sleeve member to come into contact with the spring member 70 and to force the spring member into or toward the second compressed position. The screw drive member 240 can then be rotated independently of the sleeve member 250 until the bone screw is removed. When the sleeve member is released, it is biased upwardly away from the distal end portion of the screw drive member, which allows the spring member 70 to bias back to its first relaxed position.

In an alternative embodiment shown in FIGS. 19 and 20, in a first, relaxed position, the distal end portion 244 of the screw drive member 240 is positioned within, and at least partially enclosed by, the distal end 252 of a sleeve member. In this aspect, the distal end 252 of the sleeve member has at least one recess 253 that is adapted to fit over the spring member 70 in its first, relaxed position. In this aspect, the screw removal assembly can be moved to a second engaged position, in which the sleeve member 250 is pushed upwardly longitudinally relative to the screw drive member 240, against the resistance of a bias member (not shown) that is operably positioned therebetween the sleeve member and the screw drive member. In this second position, the distal end portion 244 of the screw drive member extends outwardly and away from the distal end 252 of a sleeve member.

In use, the sleeve member 250 is placed into a seated position in the bore and is rotated to force the outside wall of the sleeve member to come into contact with the spring member and to force the spring member into or toward the second compressed position. Subsequently, the screw drive member 240 can be pushed down against the resistance of the bias member and into engagement with the head of the bone screw. The screw drive member can then be rotated independently of the sleeve member until the bone screw is removed. When the assembly is removed from the bore, the spring member is allowed to bias back to its first relaxed position.

What is claimed is:

1. A bone screw retaining system adapted to be applied the anterior human cervical spine for contacting anterior aspects of at least two cervical vertebral bodies, the bone screw retaining system comprising:
a plate that defines a plurality of transversely extending bores and a plurality of cavities, wherein the plurality of bores comprises a plurality of pairs of opposing bores, wherein each bore is counter sunk a predetermined distance and extends along a longitudinal axis between an anterior surface and a posterior surface, each bore being configured to receive a bone screw for engaging the plate to the cervical spine, wherein each bore has an upper region and a seat configured for mounting of a head of the bone screw, wherein the plurality of cavities are spaced substantially along a longitudinal axis of the plate, and wherein one cavity is positioned therebetween one pair of opposing bores such that the cavity is positioned adjacent to and equidistant from each bore of the respective pair of opposing bores; and
an elastically deformable spring member positioned therein each cavity such that portions of the spring member can extend into a portion of the upper region of each of the bores of the paired opposing bores, wherein the spring member is configured to mount therein the cavity and is movable between a first relaxed position and a second compressed position, wherein, in the first relaxed position, at least a portion of the spring member extends outwardly substantially transverse to the longitudinal axis of the bore and into the upper region of the bore, wherein, in the first relaxed position, portions of the spring member extend over portions of the upper region of each bore of the pair of opposing bores, which deceases the effective inner diameter of the upper region of the bore, and wherein, in the second compressed position, portions of the spring member are medially biased away from the longitudinal axis of the bore toward the outer wall of upper region of the bore, which increases the effective inner diameter of the upper region of the bore.

2. The bone screw retaining system of claim 1, wherein at least a portion of the cavity forms a transversely extending cavity that opens on both the posterior and anterior surfaces of the plate.

3. The bone screw retaining system of claim 1, wherein the plate has a generally elongated form having a plurality of partial lobes or lateral projections positioned at the corners and at a center of opposing longitudinally extending sides of the plate.

4. The bone screw retaining system of claim 3, wherein each partial lobe has a rounded outline and defines one respective bore.

5. The bone screw retaining system of claim 1, wherein a head of the bone screw can be configured to be posteriorly displaceable through the bore of the plate from the anterior surface to the posterior surface of the plate and is retained within a portion of the bore between the posterior and anterior surfaces.

6. The bone screw retaining system of claim 1, wherein the seat of the bore has at least a partial spherical shape.

7. The bone screw retaining system of claim 1, wherein the head of the bone screw has a maximum diameter that is smaller than the diameter of the upper region of the bore, such that the screw head can pass through the upper region of the bore.

8. The bone screw retaining system of claim 7, wherein the head of each bone screw comprises a complementary tapered section that extends outwardly therefrom a threaded shank portion of the bone screw, wherein the tapered section has a tapered surface that extends from a shank of the bone screw toward an upwardly facing shoulder surface of the bone screw which is formed by a portion of the shoulder of the head of the bone screw.

9. The bone screw retaining system of claim 8, wherein a portion of the bone screw above the upwardly facing shoulder surface of the bone screw is conventionally configured for operative engagement with a driving tool and has a reduced diameter relative to the diameter of the shoulder of the head of the bone screw.

10. The bone screw retaining system of claim 1, wherein the spring member comprises a split-ring.

11. The bone screw retaining system of claim 10, further comprising a spring mount configured to fixedly mount therein the cavity, wherein the spring member is connected thereto a portion of a side wall of the spring mount, wherein a portion of opposing side walls of the spring mount is recessed such that the upper region of the bore and the recessed edge portion of the spring mount define a generally circular countersunk well that is sized to receive the bone screw, wherein, in the first relaxed position, a portion of the spring member spans across a portion of the recessed edge portion and extends outwardly over a portion of the countersunk well substantially transverse to the longitudinal axis of the bore, wherein the spring mount has a groove defined therein the side walls of the spring mount which is configured to receive a portion of the spring member as it is medially biased toward its second compressed position when the bone screw interacts with the spring member as it is being moved posteriorly therein the bore of the plate.

12. The bone screw retaining system of claim 11, wherein opposed ends of the spring clip are enclosed therein the spring member.

13. The bone screw retaining system of claim 1, wherein the spring member is formed from a biocompatible, flexible material.

14. A bone screw retaining system adapted to be applied the anterior human cervical spine for contacting anterior aspects of at least two cervical vertebral bodies, the bone screw retaining system comprising:
a plate that defines a plurality of transversely extending bores, wherein each bore is counter sunk a predetermined distance and extends along a longitudinal axis between an anterior surface and a posterior surface, each bore being configured to receive a bone screw for engaging the plate to the cervical spine, wherein each bore has an upper region having a first diameter and a seat configured for mounting of a head of the bone screw; and
at least one spring assembly that comprises a coil spring and a piston member, wherein the spring assembly is mounted therein a portion of the upper region of the bore such that in a relaxed position, a portion of the piston member of the spring assembly extends over a portion of the upper region of the bore, wherein a portion of a wall of the upper region of the bore defines an orifice that is configured to moveably receive the coil spring and at least a portion of the piston member therein, wherein, in use, upon insertion of the bone screw into the bore of the plate and its subsequent posterior movement, a tapered surface of the head of the bone screw acts on the piston member to force the piston member medially back into orifice by compressing the underlying coil spring, and wherein, when the head of the bone screw fully engages the seat of the plate, an upwardly facing shoulder surface of the bone screw is located at or below the plane of the piston member such that the coil spring acts on the piston member and bias the piston member outwardly toward and/or to its fully extended position.

15. A bone screw retaining system adapted to be applied the anterior human cervical spine for contacting anterior aspects of at least two cervical vertebral bodies, the bone screw retaining system comprising:
a plate that defines a plurality of transversely extending bores, wherein each bore is counter sunk a predetermined distance and extends along a longitudinal axis between an anterior surface and a posterior surface, each bore being configured to receive a bone screw for engaging the plate to the cervical spine, wherein each bore has an upper region having a first diameter and a seat configured for mounting of a head of the bone screw; and
at least one elastically deformable arcuate spring member, each arcuate spring member being mounted to a portion of the upper region of the bore such that in a relaxed position, a portion of the arcuate spring member extends over a portion of the upper region of the bore substantially transverse to the longitudinal axis of the bore, wherein a portion of the wall of the upper region of the bore can define a groove that is adapted to receive a portion of the arcuate spring member as it is biased toward a second compressed position in which portions of the spring member are medially biased away from the longitudinal axis of the bore toward the wall of upper region of the bore when the bone screw is being inserted posteriorly therein the bore of the plate, wherein, in use, upon insertion of the bone screw into the bore of the plate and it subsequent posterior advancement, a tapered surface of the head of the screw acts on the arcuate spring member to force the arcuate spring member toward the second compressed position, and wherein, when the head of the bone screw fully engages the seat of the plate, an upwardly facing shoulder surface of the bone screw is located at or below the plane of the spring member such that the arcuate spring member biases toward the first relaxed position.

16. The bone screw retaining system of claim 15, wherein each respective end of the arcuate spring member is mounted therein a portion of the wall of the upper region of the bore.

17. The bone screw retaining system of claim 15, wherein a portion of the bone screw above the plane of the spring member has a reduced diameter relative to the upwardly facing shoulder surface of the bone screw.
